(19) United States
(12) Patent Application Publication
(10) Pub. No.: US 2011/0189689 A1
(43) Pub. Date: Aug. 4, 2011

(54) RIBOSWITCHES

(76) Inventors: Nenad Ban, Zurich (CH); Stéphane Thore, Nyon (CH)

(21) Appl. No.: 12/995,386

(22) PCT Filed: Jun. 1, 2009
(86) PCT No.: PCT/IB2009/006607

§ 371 Date: Apr. 18, 2011

(30) Foreign Application Priority Data
May 30, 2008 (EP) 08009940.1

(51) Int. Cl. C12Q 1/68; C07H 21/00; C07F 9/6512; C07F 9/6539; C12N 5/04; C12N 1/20; G06G 7/60

(52) U.S. Cl. 435/6.19; 536/24.1; 544/243; 435/410; 435/252.1; 703/11

(57) ABSTRACT

The TPP riboswitch is a target for antibiotics, herbicides, algicides, fungicides and other utilities. The atomic structure of the binding pocket of the TPP riboswitch has been resolved. Compounds identified and optimized using this information can be used to stimulate, activate, inhibit and/or inactivate the TPP riboswitch.

This application claims the benefit of European Application No. 08009940.1 filed May 30, 2008, the contents of which are incorporated herein by reference.

### FIELD OF THE INVENTION

The present invention generally pertains to the fields of molecular biology, regulation of gene expression, RNA crystallization, X-ray diffraction analysis, three-dimensional structure determination, structure based rational drug design, and molecular modeling of eukaryotic thiamine pyrophosphate (TPP)-specific riboswitches.

### BACKGROUND OF THE INVENTION

Precision genetic control is an essential feature of living systems, as cells must respond to a multitude of biochemical signals and environmental cues by varying genetic expression patterns. Most known mechanisms of genetic control involve the use of protein factors that sense chemical or physical stimuli and then modulate gene expression by selectively interacting with the relevant DNA or messenger RNA sequence. In addition to the widespread participation of protein factors in genetic control, it is now known that RNA can take an active role in genetic regulation.

Riboswitch RNAs are genetic control elements that are located primarily within the 5′-untranslated region (5′-UTR) of the main coding region of a particular mRNA, or sometimes in introns. Structural probing studies reveal that riboswitch elements are generally composed of two domains: a natural aptamer that serves as the ligand-binding domain, and an ‘expression platform’ that interfaces with RNA elements that are involved in gene expression (e.g. Shine-Dalgarno (SD) elements or transcription terminator stems). Riboswitches serve as metabolite-sensitive genetic switches wherein the RNA directly binds a small organic molecule. This binding process changes the conformation of the mRNA, which causes a change in gene expression by a variety of different mechanisms. The natural switches are targets for antibiotics and other small molecule therapies. Engineered riboswitches may be used as gene switches to control expression of heterologous coding regions.

Riboswitches are primarily found in bacteria. The thiamine pyrophosphate (TPP) riboswitch is found in most bacteria and in several archaea, but so far, it is the only type of riboswitch identified in eukaryotes. Thiamine pyrophosphate, commonly referred to as vitamin B1, is composed of a pyrimidine ring, a central thiazole ring and a pyrophosphate group. It is an essential metabolite in most organisms due to its role as a cofactor in a broad range of enzymatic reactions. Two thiamine analogs, oxythiamine and pyrithiamine, have been used extensively to produce thiamine deficiency in model organisms. Oxythiamine has a hydroxyl group replacing the exocyclic amino group in the pyrimidine ring, while pyrithiamine possesses a central pyridine ring rather than a thiazole ring. In the cell, these analogs are metabolized into their pyrophosphate derivatives, which inhibit many TPP-dependent enzymes. Although it has been shown that oxythiamine can reduce the growth rate of S. cerevisiae, presumably by binding to enzymes, there is no biochemical or structural information regarding the binding of oxythiamine pyrophosphate (OTPP) to the TPP riboswitch and its potential as an antibiotic. Finally, while pyrithiamine pyrophosphate (PTPP) has been shown to exert an antibiotic effect via interaction with TPP-specific riboswitches in bacteria and plants, no structural information is available for this complex.

There is a need to have a better understanding of the interaction between the TPP-specific riboswitch and its ligands. Methods and compositions that can be used to regulate TPP riboswitches would be of particular interest, e.g. for gene control in eukaryotes and as antibiotics or herbicides.

### SUMMARY OF THE INVENTION

To better understand the molecular basis of these interactions, we solved the structures of the aptamer domain of the eukaryotic TPP-specific riboswitch from Arabidopsis thaliana in complex with OTPP and PTPP at 2.65 and 2.0 Å resolution respectively. We were also able to obtain resolution for the TPP-bound riboswitch crystals at 2.25 Å, a significant improvement over our prior efforts. The aptamer domain of the TPP-specific riboswitch is highly conserved across species, so the three dimensional atomic structure should be very similar in TPP riboswitches from other species.

The coordinates and structure factors have been deposited in the Protein Data Bank with the accession number 3D2G, 3D2X and 3D2V for respectively the TPP, OTPP, and PTPP containing structures, which coordinates and structure factors are incorporated herein by reference.

We have found that there are substantial differences in the way TPP analogues are recognized by the riboswitch when compared to their enzyme binding configuration. TPP derivatives bind the riboswitch with a stretched conformation of their respective rings, i.e. the thiazole and the pyrimidine ring for OTPP or the pyridine and the pyrimidine ring for PTPP. This arrangement differs significantly from their V-shaped conformations when bound to proteins. The difference in the relative orientation of the two rings is approximately 90 degrees around the C5-C7 bond. This property permits design of riboswitch-specific TPP analogues that would efficiently target the riboswitch while not affecting the activity of enzymes that require TPP as a cofactor. Identification of these co-crystal structures thus enables the rational design and in silico screening for compounds which can act as ligands for the TPP-specific riboswitch.

The invention provides:
1.1 An isolated co-crystal of a TPP-specific riboswitch with a ligand, wherein the co-crystal is capable of resolution to 2.65 Å or less by X-ray diffraction.
1.2 The co-crystal of 1.1 wherein the ligand is selected from OTPP and PTPP.
1.3 The co-crystal of 1.1 or 1.2 having substantially the coordinates of the co-crystals described in the Protein Data Bank accession number 3D2G, 3D2X or 3D2V.
1.4 The co-crystal of any of 1.1-1.3 having substantially the cell dimensions and bond angles of the TPP bound AT riboswitch, the PTPP bound AT riboswitch OTPP bound AT riboswitch as set forth in Table 1 below.

The invention further provides
a. a computer program for depicting, modeling or analyzing the structure of a TPP-specific riboswitch, a ligand or putative ligand for a TPP-specific riboswitch, or a TPP-specific riboswitch bound to a ligand wherein the computer program comprises data substantially corresponding to the atomic coordinates for at least the ligand binding pocket of riboswitch aptamer domain of the co-crystals described in the Protein Data Bank accession number 3D2G, 3D2X and/or 3D2V;
b. a computer usable medium (e.g., CD, DVD, flash drive, hard drive, or machine readable data storage medium) having a computer readable program code comprising said computer program; and
c. a computer comprising said computer usable medium.

The invention further provides a method of identifying a compound that interacts with a TPP-specific riboswitch comprising modeling the atomic structure of the ligand binding pocket of the riboswitch aptamer domain using the above described computer program, modeling the atomic structure of a test compound, identifying test compounds that are likely to bind to the ligand binding pocket of the riboswitch aptamer domain, and optionally measuring binding of the test compounds to the riboswitch in a binding assay, and optionally further measuring interaction of the test compound with an enzyme having TPP as substrate.

The invention further provides compounds (other than TPP, OTPP and PTPP) which bind to the aptamer domain of the TPP-specific riboswitch, e.g., compounds identified using the foregoing method, and the use of such compounds to interact with a TPP riboswitch. For example the invention provides the use of Compounds of Formula I

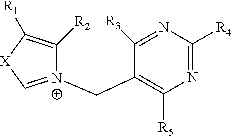

wherein

### X is S or CH═CH;

R₁ is (P₂O₆H₃)—(CH₂)ₙ— where n is 1, 2, or 3;
R₂ and R₃ are independently H or C₁₋₄alkyl, or R₂ and R₃ together form a C₂₋₃ bridge (e.g. a methylene, methylyne, ethylene or ethylyne bridge);
R₄ is H or C₁₋₄alkyl;

### R₅ is OH or NH₂;

in free base or salt form, and, where R₅ is OH, in keto or enol form; e.g., provided that the compound is other than TPP, OTPP or PTPP.

Riboswitches function to control gene expression through the binding or removal of a trigger molecule. The trigger molecule for a riboswitch (as well as other activating compounds) can be used to activate a riboswitch. Riboswitches can also be deactivated by, for example, removing trigger molecules from the presence of the riboswitch. A riboswitch can be blocked by, for example, binding of an analog of the trigger molecule that does not activate the riboswitch.

The present invention therefore discloses compounds, e.g. of Formula I as hereinbefore described, and compositions containing such compounds, that can activate, deactivate or block the TPP riboswitch, as well as compositions and methods for activating, deactivating or blocking the TPP riboswitch.

Also disclosed are compositions and methods for altering expression of an RNA molecule, or of a gene encoding an RNA molecule, where the RNA molecule includes a TPP riboswitch, by bringing a compound, e.g. of Formula I as hereinbefore described, into contact with the RNA molecule. Riboswitches function to control gene expression through the binding or removal of a trigger molecule. Thus, subjecting an RNA molecule of interest that includes a TPP riboswitch to conditions that activate, deactivate or block the riboswitch can be used to alter expression of the RNA. Expression can be altered as a result of, for example, termination of transcription or blocking of ribosome binding to the RNA. Binding of a trigger molecule or an analog thereof can, depending on the nature of the riboswitch, reduce or prevent expression of the RNA molecule or promote or increase expression of the RNA molecule.

Also disclosed are compositions and methods for regulating expression of a naturally occurring gene or RNA that contains a TPP riboswitch by activating, deactivating or blocking the riboswitch. If the gene is essential for survival of a cell or organism that harbors it, activating, deactivating or blocking the lysine riboswitch can result in death, stasis or debilitation of the cell or organism. For example, activating a naturally occurring riboswitch in a naturally occurring gene that is essential to survival of a microorganism can result in death of the microorganism (if activation of the riboswitch turns off or represses expression). This is one basis for the use of the disclosed compounds and methods for antimicrobial, antibiotic and herbicidal effects.

Disclosed herein is a method of inhibiting gene expression, the method comprising bringing into contact a compound, e.g. of Formula I as hereinbefore described, and a cell. The cell can be identified as being in need of inhibited gene expression. The cell can be a plant cell, for example, and the compound can kill or inhibit the growth of the plant cell. The compound and the cell can be brought into contact by administering the compound to a subject. In one example, the compound is not a substrate for enzymes of the subject that have TPP as a substrate.

Further disclosed is a composition comprising the compound described above and a regulatable gene expression construct comprising a nucleic acid molecule encoding an RNA comprising a TPP riboswitch operably linked to a heterologous coding region, wherein the TPP riboswitch regulates expression of the RNA, wherein the TPP riboswitch and coding region are heterologous. The TPP riboswitch can produce a signal when activated by the compound. For example, the riboswitch can change conformation when activated by the compound, and the change in conformation can produce a signal via a conformation dependent label. Furthermore, the riboswitch can change conformation when activated by the compound, wherein the change in conformation causes a change in expression of the coding region linked to the riboswitch, wherein the change in expression produces a signal. The signal can be produced by a reporter protein expressed from the coding region linked to the riboswitch.

Also disclosed is a method comprising: (a) testing the compound as described above for inhibition of gene expression of a gene encoding an RNA comprising a TPP riboswitch, wherein the inhibition is via the TPP riboswitch, and (b) inhibiting gene expression by bringing into contact a cell and a compound that inhibited gene expression in step (a), wherein the cell comprises a gene encoding an RNA comprising the TPP riboswitch, wherein the compound inhibits expression of the gene by binding to the TPP riboswitch.

Further disclosed is a method of inhibiting the growth of and/or killing bacteria, yeast, algae or higher plants, comprising contacting the organism with a compound disclosed above, for example the use of a compound identified through the methods of identification described above, or a compound of Formula I as described above, as a herbicide, algicide, antifungal or antibacterial.

Disclosed herein is also a method of inhibiting growth of a cell, such as a bacterial or fungal cell, that is in a mammalian subject, the method comprising administering an effective amount of a compound as disclosed, e.g., of Formula 1, to the subject. This can result in the compound being brought into contact with the cell. The subject can have, for example, a bacterial infection, and the bacterial cells can be the cells to be inhibited by the compound. The bacteria can be any bacteria having the TPP riboswitch. Bacterial growth can also be inhibited in any context in which bacteria are found. For example, bacterial growth in fluids, biofilms, and on surfaces can be inhibited. The compounds disclosed herein can be administered or used in combination with any other compound or composition. For example, the disclosed compounds can be administered or used in combination with another antimicrobial compound.

Also disclosed are compositions and methods for selecting and identifying compounds that can activate, deactivate or block a riboswitch. Activation of a riboswitch refers to the change in state of the riboswitch upon binding of a trigger molecule. A riboswitch can be activated by compounds other than the trigger molecule and in ways other than binding of a trigger molecule. The term trigger molecule is used herein to refer to molecules and compounds that can activate a riboswitch. This includes the natural or normal trigger molecule for the riboswitch and other compounds that can activate the riboswitch. Natural or normal trigger molecules are the trigger molecule for a given riboswitch in nature or, in the case of some non-natural riboswitches, the trigger molecule for which the riboswitch was designed or with which the riboswitch was selected (as in, for example, in vitro selection or in vitro evolution techniques). Non-natural trigger molecules can be referred to as non-natural trigger molecules.

Deactivation of a riboswitch refers to the change in state of the riboswitch when the trigger molecule is not bound. A riboswitch can be deactivated by binding of compounds other than the trigger molecule and in ways other than removal of the trigger molecule. Blocking of a riboswitch refers to a condition or state of the riboswitch where the presence of the trigger molecule does not activate the riboswitch. Activation of a riboswitch can be assessed in any suitable manner. For example, the riboswitch can be linked to a reporter RNA and expression, expression level, or change in expression level of the reporter RNA can be measured in the presence and absence of the test compound. As another example, the riboswitch can include a conformation dependent label, the signal from which changes depending on the activation state of the riboswitch. Such a riboswitch preferably uses an aptamer domain from or derived from a naturally occurring riboswitch. As can be seen, assessment of activation of a riboswitch can be performed with the use of a control assay or measurement or without the use of a control assay or measurement. Methods for identifying compounds that deactivate a riboswitch can be performed in analogous ways.

Also disclosed are compounds made by identifying a compound that activates, deactivates or blocks a riboswitch and manufacturing the identified compound. This can be accomplished by, for example, combining compound identification methods as disclosed elsewhere herein with methods for manufacturing the identified compounds. For example, compounds can be made by bringing into contact a test compound and a riboswitch, assessing activation of the riboswitch, and, if the riboswitch is activated by the test compound, manufacturing the test compound that activates the riboswitch as the compound.

Also disclosed are compounds made by checking activation, deactivation or blocking of a riboswitch by a compound and manufacturing the checked compound. This can be accomplished by, for example, combining compound activation, deactivation or blocking assessment methods as disclosed elsewhere herein with methods for manufacturing the checked compounds. For example, compounds can be made by bringing into contact a test compound and a riboswitch, assessing activation of the riboswitch, and, if the riboswitch is activated by the test compound, manufacturing the test compound that activates the riboswitch as the compound. Checking compounds for their ability to activate, deactivate or block a riboswitch refers to both identification of compounds previously unknown to activate, deactivate or block a riboswitch and to assessing the ability of a compound to activate, deactivate or block a riboswitch where the compound was already known to activate, deactivate or block the riboswitch.

Disclosed herein is also a method of inhibiting growth of a cell, such as a bacterial cell, that is in a subject, the method comprising administering an effective amount of a compound as disclosed herein to the subject. This can result in the compound being brought into contact with the cell. The subject can have, for example, a bacterial infection, and the bacterial cells can be the cells to be inhibited by the compound. The bacteria can be any bacteria, such as bacteria from the genus Bacillus, Actinobacillus, Clostridium, Desulfitobacterium, Enterococcus, Erwinia, Escherichia, Exiguobacterium, Fusobacterium, Geobacillus, Haemophilus, Idiomarina, Lactobacillus, Lactococcus, Leuconostoc, Listeria, Moorella, Oceanobacillus, Oenococcus, Pasteurella, Pediococcus, Shewanella, Shigella, Solibacter, Staphylococcus, Thermoanaerobacter, Thermotoga, and Vibrio, for example. Bacterial growth can also be inhibited in any context in which bacteria are found. For example, bacterial growth in fluids, biofilms, and on surfaces can be inhibited. The compounds disclosed herein can be administered or used in combination with any other compound or composition. For example, the disclosed compounds can be administered or used in combination with another antimicrobial compound.

### DETAILED DESCRIPTION OF THE INVENTION

Thiamine pyrophosphate (TPP)-specific riboswitch is a widespread genetic element regulating the availability of this essential vitamin. It is so far the only riboswitch found in eukaryotic cells. Furthermore, the TPP-binding domain is an attractive target for anti bacterial and anti fungal compounds. We have solved the crystal structure of the Arabidopsis thaliana TPP-binding domain of the TPP-specific riboswitch, highly homologous in all the TPP-specific riboswitches, at a resolution of 2.0 Å for the complex with Pyrithiamine Pyrophosphate, 2.5 Å for complex with Oxythiamine Pyrophosphate and 2.5 Å for complex with TPP at 2.25 Å), and refined it to better than 26% free R-factor. The interpretation of the electron density with atomic coordinates reveals detailed structural information on the sites responsible for TPP and two relative compounds, pyrithiamine pyrophosphate and oxythiamine pyrophosphate, binding and the structural domains that are regulating gene expression. The eukaryotic TPP-binding riboswitch adopts a Y-shape organized around a three way junctions. Two helices are responsible for binding the small molecules. The respective sites of binding for the thiamine derived compounds are oriented towards the center of the Y-shaped molecule. These observations, together with the range of conformations observed in this crystallographic study, provide detailed chemical information on these complexes with important implications for drug design.

The current invention provides crystals of Arabidopsis thaliana TPP-binding domain of the riboswitch bound to TPP, Pyrithiamine pyrophosphate (PTPP) and Oxythiamine pyrophosphate (OTPP) as well as crystallographic data from which atomic models of Arabidopsis thaliana TPP-binding domain of the riboswitch bound to these compounds were determined. Arabidopsis thaliana TPP-binding domain of the riboswitch is highly homologous to all other TPP-binding domain of riboswitch systems. The atomic model for Arabidopsis thaliana TPP-binding domain of the riboswitch thus provides detailed information to model structures of evolutionary related domains, including, but not limited to, those which contain two or more TPP-binding homologous domains. Such modeled structures are useful to interpret low resolution structural data for any of the aforementioned related RNA elements and to predict or assess the molecular details of binding of small-molecular or macromolecular ligands, which modulate the biological activity of these RNA elements.

The current invention includes the growth of crystals of Arabidopsis thaliana TPP-binding domain of the TPP-specific riboswitch to a maximum size of 0.5×0.2×0.1 mm (cf. FIG. 1) and in particular of crystals with dimensions larger than 0.2×0.1×0.05 mm. The current invention also includes the collection of X-ray crystallographic data from such crystals, in particular the collection of crystallographic data to a resolution of at least 2.0 Å for the PTPP-bound form of the TPP-specific riboswitch and the calculation of an experimentally phased electron density map at a resolution of at least 2.9 Å resolution. The invention also includes the three dimensional structure of TPP-binding domain of the A. thaliana TPP-specific riboswitch in complex with PTPP refined to 2.0 Å resolution, the three dimensional structure of TPP-binding domain of the A. thaliana TPP-specific riboswitch in complex with TPP refined to 2.25 Å resolution and the three dimensional structure of TPP-binding domain of the A. thaliana TPP-specific riboswitch in complex with OTPP refined to 2.5 Å resolution. The structure contains atomic coordinates for the complete TPP-binding domain of the TPP-specific riboswitch responsible for TPP binding and atomic coordinates of the three different bound ligands, TPP, PTPP and OTPP respectively.

The refined model of the TPP-bound riboswitch contains 163 water molecules and 10 magnesium (Mg²⁺) ions. Overall, it is similar to our earlier lower resolution structure except for the conformation of the TPP pyrophosphate moiety, where the higher resolution map reveals an extended conformation bound to two Mg²⁺ ions and a closely coordinated network of water molecules. The observed conformation of the pyrophosphate and its interaction with the two Mg²⁺ ions is similar to the one observed in the structures of the bacterial TPP-specific riboswitch.

While the overall structure of the riboswitch in complex with OTPP or PTPP is similar to its structure in complex with the natural ligand, important differences emerge in the binding interactions of these analogs.

The structure of the OTPP complex reveals that the interactions between the pyrophosphate moiety and the riboswitch are near identical to those observed for TPP. One might have predicted that the substitution of the exocyclic amino group of TPP by a keto group at the C4 position of the pyrimidine ring of OTPP would lead to a different mode of interaction with the ligand-binding pocket. Instead, the OTPP molecule interacts with the purine ring of G28 with the same geometry as observed for the TPP pyrimidine ring. Since the most favored tautomeric form of the OTPP in solution has the N3 position protonated, the actual mode of binding implies that the pyrimidine ring of the OTPP is stabilized in its enol form. Therefore, the structure supports the biochemical data indicating that the oxythiamine, the non-phosphorylated form of the thiamine analog, displayed a strongly reduced affinity to the E. col±165 thiM RNA riboswitch as the binding of the enol tautomer is energetically disfavored.

The specific contacts between the RNA and the pyrophosphate and pyrimidine moieties of the antimicrobial PTPP observed in the structure of its complex with the riboswitch are the same as in the case of TPP riboswitch complex. Nevertheless, the absence of the sulfur atom in the pyridine ring of PTPP reduces the acidic character of the central ring and permits closer interactions with the base of G60. This nucleotide base is not well ordered in the TPP or OTPP-containing structures but becomes clearly visible in the PTPP-bound riboswitch structure, where it stacks against the edge of the PTPP central ring. This conformation of G60 is further stabilized by water mediated hydrogen bonds with the PTPP alpha phosphate.

There are some substantial differences in the way TPP analogues are recognized by the riboswitch when compared to their enzyme binding mode. TPP derivatives bind the riboswitch with a stretched conformation of their respective rings, i.e. the thiazole and the pyrimidine ring for OTPP or the pyridine and the pyrimidine ring for PTPP. This arrangement differs significantly from their V-shaped conformations when bound to proteins. The difference in the relative orientation of the two rings is approximately 90 degrees around the C5-C7 bond.

This property could be explored to synthesize riboswitch-specific TPP analogues that would efficiently target the riboswitch while not affecting the activity of enzymes that require TPP as a cofactor.

The crystal structures of oxythiamine pyrophosphate and pyrithiamine pyrophosphate in complex with the A. thaliana TPP-specific riboswitch, described here, provide detailed chemical information on these complexes with important implications for drug design. The OTPP riboswitch complex reveals that the pyrithiamine ring of OTPP is stabilized in its enol form when bound to the riboswitch. The structure of PTPP, a well characterized antimicrobial, in complex with the riboswitch highlights new interactions possibly leading to tighter binding pocket as exemplified by the rotation of the base of Guanosine 60.

### Example 1

### Preparation and Analysis of Co-Crystals

Synthesis and Purification: PTPP is synthesized using conventional means. Briefly, ortho-phosphoric acid (0.5 g, Fluka #79622) is liquefied over flame and cooled to room temperature. Pyrithiamine hydrobromide (5 mg, Sigma #P0256) is then added and the mixture is stirred for 15 min in an oil bath heated to 110° C. After cooling, the reaction is quenched by adding 2 ml of water and magnesium oxide powder until the pH value reached 7.0. This step allows the removal of the unreacted inorganic phosphate for further purification using a C18 column (Vydac 218TP 54 or 1022) with TriEthylAmmonium solution at 100 mM (pH 7.0). Pyrithiamine Mono, Di and Tri-phosphate can easily be separated by the HPLC chromatography. Fractions containing the PTPP are collected and concentrated. An identical procedure is used for the synthesis of OTPP starting from oxythiamine chloride hydrochloride (Sigma #O4000).

TPP-binding domain of the A. thaliana TPP-specific riboswitch is synthesized using general methods for the in vitro synthesis of RNA molecules. In detail, the synthesis involves obtaining large quantities of DNA with the sequence of the TPP-binding domain preceded by a T7 promoter. The DNA is in vitro transcribed with T7 RNA polymerase for 3 h at 37° C. After 2 acidic phenol extractions, the RNA is precipitated and resuspended in water. Single nucleotides and salt are removed by centrifugation with a Vivaspin centricon (10K molecular weight cut off).

The RNA is concentrated to 0.4 mM (approximately 10 mg/ml) and annealed for 5 min at 95° C. in the presence of 1 mM of the respective compounds, i.e. TPP, PTPP or OTPP. After 10 min on ice, the RNA is diluted twice with crystallization buffer (10 mM Hepes pH7.0, 50 mM NaCl, 5 mM MgCl) and used immediately for crystallization or stored at −80° C.

Crystallization: Equal volumes between 0.5 μl and 2 μl of concentrated small molecule/RNA solution and a reservoir solution consisting of 8-12% (v/v) 1,6-hexanediol, 5-15 mM magnesium sulfate, 0-1 mM spermine and 40 mM sodium cacodylate, pH 6.6-7.2 are mixed in the well of a sitting drop crystallization plate and equilibrated against 500 μl of the reservoir solution by vapor diffusion. Initial crystals appear within 24 hours, crystals of a maximal size of 0.5×0.2×0.1 mm grow within two weeks. For crystallographic data collection, the solution around the crystals is gradually exchanged against a solution similar to the reservoir solution with the following difference: 1.5 mM TPP, PTPP or OTPP and 25-30% (v/v) 1,6-hexanediol.

The analog/riboswitch crystalline complexes are solved the structures by molecular replacement. Diffraction data are collected at beamline X06SA of the Swiss Light Source (Villigen, Switzerland) and processed with XDS (Kabsch, W. J. Appli. Cryst. 1993, 26, 795-800) for OTPP and PTPP or HKL2000 (Otwinowski, Z.; Minor, W. Meth. Enzym. 1997, 276, 307-326) for TPP. Subsequent rounds of rigid body and simulated annealing with positional and B-factor refinement are done in CNS (Brunger, A. T.; Adams, P. D.; Clore, G. M.; DeLano, W. L.; Gros, P.; Grosse-Kunstleve, R. W.; Jiang, J. S.; Kuszewski, J.; Nilges, M.; Pannu, N. S.; Read, R. J.; Rice, L. M.; Simonson, T.; Warren, G. L. Acta Crystallogr. D Biol. Crystallogr. 1998, 54, (Pt 5), 905-21) for the OTPP/RNA structure and with the PHENIX software (Adams, P. D.; Gopal, K.; Grosse-Kunstleve, R. W.; Hung, L. W.; Ioerger, T. R.; McCoy, A. J.; Moriarty, N. W.; Pai, R. K.; Read, R. J.; Romo, T. D.; Sacchettini, J. C.; Sauter, N. K.; Storoni, L. C.; Terwilliger, T. C. J. Synchrotron Radiat. 2004, 11, (Pt 1), 53-5) for the TPP/RNA and PTPP/RNA complexes. Refined models are obtained with final crystallographic working R factors and free-R factors of 20.9/25.7, 23.3/26.9 and 20.7/25.1, for the TPP, OTPP and PTPP-bound A. thaliana riboswitch, respectively.

TABLE 1
Crystallographic Statistics

| | TPP bound AT | | PTPP bound AT | | OTPP bound AT | |
|---|---|---|---|---|---|---|
| | riboswitch | | riboswitch | | riboswitch | |
| Data collection | | | | | | |
| X-ray source | SLS X06SA | | SLS X06SA | | SLS X06SA | |
| Space group | P2₁2₁2 | | P2₁2₁2 | | P2₁2₁2 | |
| Cell dimensions | | | | | | |
| a, b, c (Å) | 76.0, 111.0, 55.8 | | 74.8, 110.8, 55.8 | | 75.5, 111.2, 55.4 | |
| □, □, □ | 90, 90, 90 | | 90, 90, 90 | | 90, 90, 90 | |
| Wavelength (Å) | 1.0 | | 1.0 | | 1.0 | |
| Resolution (Å)* | 15-2.25 | (2.31-2.25) | 50-1.97 | (2.02-1.97) | 15.0-2.5 | (2.55-2.50) |
| R_{merge} (%)* | 7.5 | (97.2) | 4.8 | (40.7) | 15.3 | (53.3) |
| <I/□□I)>* | 23.45 | (2.0) | 33.5 | (5.87) | 14.63 | (2.85) |
| Completeness (%)* | 100.0 | (100.0) | 98.8 | (92.7) | 97.2 | (97.1) |
| Redundancy* | 7.1 | (7.2) | 7.1 | (5.3) | 7.3 | (7.5) |

| | | | |
|---|---|---|---|
| Refinement | | | |
| Resolution (Å) | 15-2.25 | 15.0-2.00 | 15.0-2.50 |
| No. reflections | 22939 | 33,016 | 16,268 |
| R_{work}/R_{free} (%) | 20.9/25.7 | 20.7/25.1 | 23.3/26.7 |
| R.m.s. deviations | | | |
| Bond lengths (Å) | 0.017 | 0.003 | 0.006 |
| Bond angles (°) | 3.16 | 0.857 | 1.100 |
| □A coordinate error | 0.35 | 0.28 | 0.37 |
| (Å) | | | |

*Highest resolution shell in ( ).

Subsequent to the priority date of this application, portions of the work described herein were published by Thore, S., et al. “Structural basis of thiamine pyrophosphate analogues binding to the eukaryotic riboswitch” (2008) J. Am. Chem. Soc. 130: 8116-8117, the contents of which article, including the on-line supplemental materials, are incorporated herein by reference.

1. An isolated co-crystal of a thiamine pyrophosphate (TPP) riboswitch with a ligand, wherein the co-crystal is capable of resolution to 2.65 Å or less by X-ray diffraction.

2. The co-crystal of claim 1 wherein the ligand is selected from pyrithiamine pyrophosphate (PTPP) and oxythiamine pyrophosphate (OTPP).

3. The co-crystal of claim 1 having substantially the coordinates of the co-crystals described in the Protein Data Bank accession number 3D2G, 3D2X or 3D2V.

4. The co-crystal of claim 1 having substantially the cell dimensions and bond angles of the TPP bound AT riboswitch, the PTPP bound AT riboswitch OTPP bound AT riboswitch as set forth in Table 1.

5. A computer program for depicting, modeling or analyzing the structure of a TPP-specific riboswitch, a ligand or putative ligand for a TPP-specific riboswitch, or a TPP-specific riboswitch bound to a ligand wherein the computer program comprises data substantially corresponding to the atomic coordinates for at least the ligand binding pocket of riboswitch aptamer domain of the co-crystals described in the Protein Data Bank accession number 3D2G, 3D2X and/or 3D2V.

6. A computer usable medium having a computer readable program code comprising the computer program of claim 5.

7. A computer comprising the computer usable medium of claim 6.

8. A method of identifying a compound that interacts with a TPP-specific riboswitch comprising modeling the atomic structure of the ligand binding pocket of the riboswitch aptamer domain using the computer program of claim 5, modeling the atomic structure of a test compound, identifying test compounds that are likely to bind to the ligand binding pocket of the riboswitch aptamer domain, and optionally measuring binding of the test compounds to the riboswitch in a binding assay.

9. A method of activating, deactivating or blocking a TPP riboswitch comprising providing to a cell expressing a TPP riboswitch a Compound of Formula I 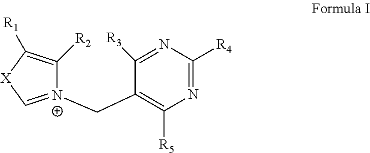
wherein
X is S or CH═CH;
R₁ is (P₂O₆H₃)—(CH₂)ₙ— where n is 1, 2, or 3;
R₂ and R₃ are independently H or C₁₋₄alkyl, or R₂ and R₃ together form a C₂₋₃ bridge;
R₄ is H or C₁₋₄alkyl;
R₅ is OH or NH₂;
in free base or salt form, and where R₅ is OH, in keto or enol form;
wherein the compound is other than TPP, OTPP or PTPP.

10. The method of claim 9 wherein the cell is a plant cell or a bacterial cell.

11. A method of altering expression of an RNA molecule, or of a gene encoding an RNA molecule, where the RNA molecule includes a TPP riboswitch, by bringing a Compound of Formula I according to claim 9 into contact with the RNA molecule.

12. A herbicide, algicide, antifungal or antibacterial composition comprising the Compound of Formula I.
